(19) United States
(12) Patent Application Publication
(10) Pub. No.: US 2026/0322137 A1
(43) Pub. Date: Oct. 15, 2026

(54) WIRELESS POWER FOR BURIED ELECTRONICS

(71) Applicant: University of Florida Research Foundation, Inc., Gainesville, FL (US)

(72) Inventors: David P. Arnold, Gainesville, FL (US); Vernon Crasto, Gainesville, FL (US)

(21) Appl. No.: 19/699,415

(22) Filed: Jun. 5, 2026

Related U.S. Application Data
(63) Continuation of application No. PCT/US2024/058826, filed on Dec. 6, 2024.
(60) Provisional application No. 63/607,764, filed on Dec. 8, 2023.

(51) Int. Cl. H02J 7/16; G01N 33/24; H01M 10/44; H02J 50/40; H02K 21/14

(52) U.S. Cl. CPC H02J 7/163; G01N 33/246; H01M 10/441; H02J 50/40; H02K 21/14

(57) ABSTRACT

The present disclosure presents wireless power transmission systems and related methods. One such system comprises an above-ground wireless power transmitter comprising a waveform generator and a coil transmitter that is configured to generate a time-varying field; and an underground electromechanical receiver comprising a permanent magnet that oscillates in response to the time-varying field that results in electricity being generated using electrodynamic transduction and being supplied to an underground electrical device that is coupled to the underground electromechanical receiver.

### CROSS-REFERENCE TO RELATED APPLICATION

This application is a continuation of co-pending PCT Application No. PCT/US2024/058826, filed Dec. 6, 2024, which claims the benefit of and priority to U.S. provisional application entitled, “Wireless Power For Buried Electronics,” having application No. 63/607,764, filed Dec. 8, 2023, each of which is entirely incorporated herein by reference.

### STATEMENT REGARDING FEDERALLY SPONSORED RESEARCH OR DEVELOPMENT

This invention was made with government support under Grant No. 1941529 awarded by the National Science Foundation. The government has certain rights in the invention.

### BACKGROUND

Wireless power transfer (WPT) is an attractive method to recharge battery-powered devices (e.g., mobiles, wearables, UAVs (unmanned aerial vehicles), IoT (Internet-of-Things) sensors and more) and eliminates the burden of plugging in a charging cable. Transferring useful power level with relatively good efficiency to compact receivers over extended distances (no contact) is the main WPT challenge. For example, commonly used inductive WPT systems face unavoidable technical and safety concerns regarding the use of electromagnetic fields between the transmitting and receiving systems.

### SUMMARY

Embodiments of the present disclosure provide wireless power transmission systems and related methods. One such system comprises an above-ground wireless power transmitter comprising a waveform generator and a coil transmitter that is configured to generate a time-varying field; and/or an underground electromechanical receiver comprising a permanent magnet that oscillates in response to the time-varying field that results in electricity being generated using electrodynamic transduction and being supplied to an underground electrical device that is coupled to the underground electromechanical receiver.

Accordingly, the present disclosure can also be viewed as providing wireless power transmission methods. In this regard, one embodiment of such a method, among others, can be broadly summarized by the following steps: coupling a pickup coil of an electromechanical receiver to a rechargeable battery, wherein the electromechanical receiver further comprises a permanent magnet that oscillates in response to a time-varying field that results in electricity being generated using electrodynamic transduction and being supplied to the rechargeable battery via the pickup coil; burying the electromechanical receiver and the rechargeable battery underground; generating the time-varying field with an above-ground wireless power transmitter; and/or charging the rechargeable battery that is buried underground.

In one or more aspects for such systems or methods, an exemplary system/method can involve a plurality of underground electromechanical receivers, wherein the plurality of underground electromechanical receivers include the underground electromechanical receiver; the underground electrical device comprises a rechargeable battery; the underground electrical device comprises a soil moisture sensor; the underground electromechanical receiver has five-phase windings; the underground electromechanical receiver has three-phase windings; the above-ground wireless power transmitter is mounted on a vehicle that is configured to traverse a field, wherein the underground electromechanical receiver is buried in the field; the time-varying field is transmitted over a distance of 10 cm to 30 cm from the above-ground wireless power transmitter to the underground electromechanical receiver; and/or the underground electromechanical receiver further comprises a pickup coil and a rechargeable battery, wherein oscillation of the permanent magnet induces an alternating voltage waveform in the pickup coil that is input to the rechargeable battery.

In one or more aspects, such methods may further comprise powering an underground sensor with the rechargeable battery; powering a wireless communication module with the rechargeable battery; transmitting sensor readings from the underground sensor using the wireless communication module to an above ground communication gateway device; and/or mounting the above-ground wireless power transmitter on a vehicle that traverse a field, wherein the electromechanical receiver is buried in the field.

Other systems, methods, features, and advantages of the present disclosure will be or become apparent to one with skill in the art upon examination of the following drawings and detailed description. It is intended that all such additional systems, methods, features, and advantages be included within this description and be within the scope of the present disclosure.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows an exemplary electrodynamic wireless power transmission (EWPT) system in accordance with various embodiments of the present disclosure.

FIGS. 2-3 show exemplary EWPT systems deployed in an agricultural environment in accordance with various embodiments of the present disclosure.

FIG. 4 shows a circuit schematic for an exemplary three-phase receiver of the EWPT system in accordance with various embodiments of the present disclosure.

FIG. 5 shows a circuit schematic of receiver components of an exemplary EWPT system in accordance with various embodiments of the present disclosure.

FIG. 6 provides a chart showing the maximum power output for various configurations of the three-phase receiver of FIG. 4 in accordance with various embodiments of the present disclosure.

FIG. 7A-7E provides charts showing increases in battery voltage during different sections of a charging time of the three-phase receiver of FIG. 4.

FIG. 8 provides a chart showing voltage and current consumption of an exemplary wireless communication module during a discharging cycle of an exemplary receiver system in accordance with various embodiments of the present disclosure

FIG. 9 shows a dashboard interface of a soil moisture reading for an exemplary EWPT system in accordance with various embodiments of the present disclosure.

FIG. 10 shows a schematic of an exemplary EWPT system with three five-phase receivers in accordance with various embodiments of the present disclosure.

FIGS. 11A-11C show circuit schematics of three different loading configurations for the five-phase receivers of FIG. 10.

FIG. 12 shows an equivalent circuit model of an exemplary five-phase receiver of FIG. 10.

FIG. 13 provides a chart showing a simulation of total load power from the equivalent circuit model of an exemplary five-phase receiver of FIG. 12.

FIG. 14A provides a chart showing average power delivered by an exemplary receiver across resistive loads for the three different loading configurations of FIGS. 11A-11C.

FIG. 14B provides a chart showing DC output power across resistive loads for a one receiver setup versus a three receiver setup of an exemplary EWPT system in accordance with various embodiments of the present disclosure.

FIG. 14C provides a chart showing pull-out frequencies for various loading configurations of FIGS. 11A-11C.

FIGS. 15A-15D provides charts showing wireless power transfer characterization of an electromagnet transmitter in comparison with an air-core transmitter, in accordance with the present disclosure, in terms of pull-out frequency, average power, power transfer efficiency, and off-axis power transfer capability, respectively.

FIG. 16 is an exemplary EWPT system of the present disclosure with a rotating magnetic receiver being driven either by (a) a coil transmitter, (b) a rotating magnet transmitter, or (c) an electromagnet transmitter, in accordance with various embodiments of the present disclosure.

FIG. 17A shows a schematic of a dual coil transmitter system with alternate polarity to power a receiver, in accordance with various embodiments of the present disclosure.

FIGS. 17B-17C are charts comparing performances of a dual coil transmitter and a single coil transmitter, in accordance with the present disclosure, in terms of output power and efficiency, respectively.

### DETAILED DESCRIPTION

In accordance with various embodiments of the present disclosure, an exemplary rotating-magnet electrodynamic wireless power transmission (EWPT) system is used to power underground electronics (e.g., a soil moisture sensor or sensor network). Accordingly, the needs for wirelessly recharging battery powered infrastructure have been on an upward trajectory over the past decade. Electrodynamic wireless power transfer (EWPT) differs from inductive wireless power transfer (IPT) by employing a mechanically responsive permanent magnet in a receiver, as opposed to an electromagnetically responsive (inductive) coil. EWPT offers the ability of transferring energy at increased distances while still achieving significant power transfer efficiencies. Due to lower operating frequencies (<1 kHz), EWPT power transfer is less influenced by the presence of conductive materials and can leverage higher amplitude magnetic fields while remaining below human exposure limits.

One example use case is for powering buried sensors/communication platforms in an agricultural environment. In various embodiments, an exemplary EWPT system of the present disclosure comprises at least one transmitter and at least one receiver. In various embodiments, the transmitter is mounted on moving infrastructure (e.g. a tractor, irrigation system, or robot) that traverses through a field, and when the transmitter passes over a buried receiver coupled to underground electronics, power is delivered to the buried system (e.g., receiver and electronics). Such a system and related methods enable the deployment and energy sustainment of underground sensor networks.

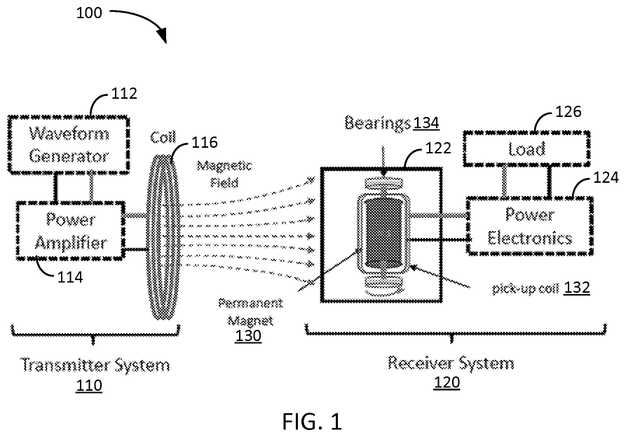

FIG. 1 shows an exemplary EWPT system 100 in accordance with various embodiments of the present disclosure. The system 100 includes a transmitter system 110 having a waveform generator 112, a power amplifier 114, and a transmitting coil 116. Correspondingly, the system 100 also includes a receiver system 120 having a permanent magnet assembly 122 coupled to power electronics 124 and load 126 (e.g., sub-surface sensor(s)), where the permanent magnet assembly includes a permanent magnet 130, pickup coil 132, and bearing 134 components. Accordingly, in various embodiments, the receiver system 120 comprises an electromechanical receiver that potentially overcomes the technical and safety concerns that are not able to be overcome by inductive WPT systems. In an exemplary EWPT system, a permanent magnet in the EWPT receiver system 120 oscillates (translational or rotational) when subjected to a time-varying field supplied from the transmitter 110 which is then converted into electricity using electrodynamic (ED) transduction, as illustrated in the figure.

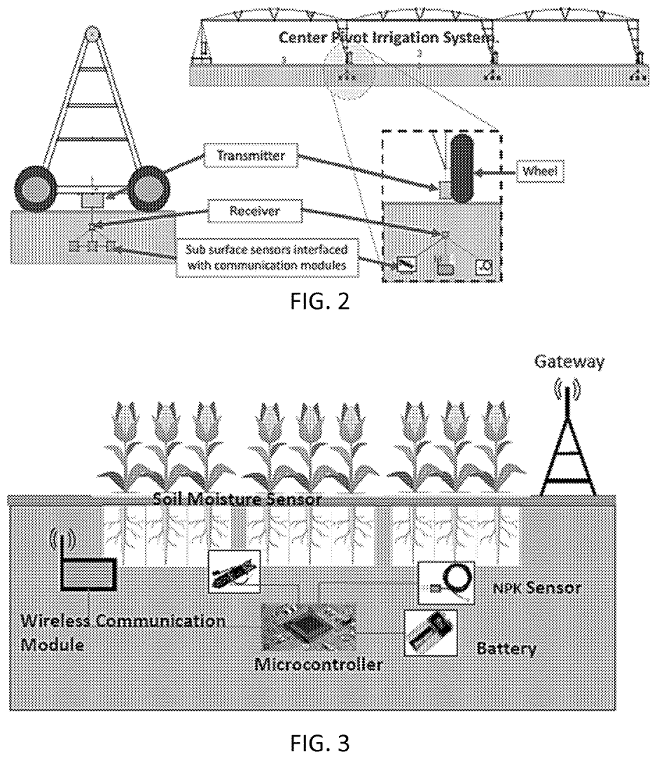

In general, Internet-of-Things (IoT) based field and crop health monitoring-systems contain end devices equipped with a variety of sensors and communication modules that require uninterrupted power source for continuous operation. Conventional batteries usually meet the need, but they require frequent replacement/recharging (due to short life-span) which is inconvenient, or sometimes, impossible. However, an exemplary wireless power transfer system of the present disclosure is capable of recharging batteries in a cluttered environment (e.g., in presence of conductive media (plants, soil, metal enclosures etc.)) and transmitting a significant amount of power with minimal attenuation to one or more EWPT receivers 120 embedded with sensors and communication nodes at a distance through clutters, as demonstrated in FIG. 2.

Here, an irrigation system is equipped with a transmitter 110 that communicates with a buried receiver 120 that is coupled to sub-surface sensors as part of an underground sensor network. Accordingly, as the irrigation system traverses through a field, when the transmitter 110 passes over the buried receiver 120 that is coupled to underground electronics, power is delivered to the buried system (e.g., receiver and electronics). Such a system (and related methods) enable the deployment and energy sustainment of underground sensor networks.

As demonstrated in FIG. 3, the underground sensor network may be a soil moisture sensor network, in various embodiments, where the underground electronics can include a wireless communication module, microcontroller, secondary battery, and a soil sensor configured to detect nitrogen, phosphorus, and potassium content in the soil (NPK sensor), such that sensor readings can be communicated from the wireless communication module to an above ground gateway communication device.

In one embodiment, an exemplary buried sensor network system comprises an EWPT receiver 120, a rectifier circuit, a battery management process performed by a microcontroller, a secondary battery in the form of a rechargeable Li-ion coin cell battery, a wireless communication module in the form of an ESP-8266 Wi-Fi SoC, and a buried sensor in the form of a capacitive soil moisture sensor. During a charging cycle for such a system, the sub-surface EWPT receiver 120 is configured to harvest energy from an electromagnetic field supplied by the transmitter 110 above the ground. The permanent magnet in the receiver system 120 interacts with the field from the transmitter coil 116, and gradually increases its angular velocity to achieve steady state rotation. The steady state motion of the permanent magnet 130 induces an alternating voltage in the multiphase pick-up windings of the pickup coil 132 of the receiver 120. The alternating waveform is rectified and filtered before feeding it into the microcontroller's battery management system which monitors the voltage on the rechargeable coin cell battery and regulates the charging current.

During a discharging cycle, in various embodiments of FIG. 3, the fully charged battery powers the ESP-8266 Wi-Fi SoC and the capacitive soil moisture sensor. The ESP-8266 wireless communication module reads the analog data from the soil moisture sensor and is programmed to upload data at regular intervals using the message queuing telemetry transport (MQTT) protocol, a lightweight messaging protocol ideal for IoT applications via an Adafruit IO platform. In various embodiments, the ESP-8266 Wi-Fi SoC enters deep sleep mode between data transmissions to conserve power.

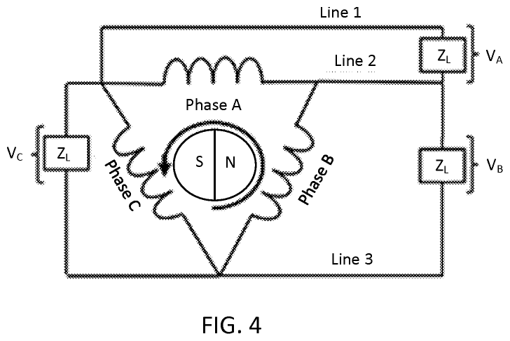

In various embodiments, the buried receiver system 120 comprises a multiphase EWPT receiver. For example, FIG. 4 shows a circuit schematic for an exemplary three-phase receiver. In certain non-limiting implementations, the three-phase receiver (Receiver A) is produced using 1″ diameter 1″ long cylindrical permanent magnets supported on a shaft and ceramic bearings with three-phase pickup coils to maximize power output. Such a receiver was tested at distances from 10 cm to 30 cm (~1 foot) with a transfer of significant power figures at extended distances with considerable efficiency having been achieved. Power transfer figures of 10 W at 10 cm and up to 975 mW at 30 cm with efficiencies of 72% and 7.5%, respectively, were demonstrated. The operating range of the receiver was <1 kHz with 10 W of power produced at a maximum speed of 461 Hz.

In an alternative implementation, the EWPT system 100 is comprised of a coil-based transmitter 110 and a receiver 120 (Receiver B) featuring a diametrically magnetized permanent magnet (PM) 130 that rotates within the pick-up windings 132. The coil transmitter 110 comprises a pancake-shaped coil with 350 turns of AWG 12 copper wire, measuring 30 cm in outer diameter, 10.8 cm in inner diameter, and 1.7 cm in length. The receiver 120 comprises a cylindrical, diametrically magnetized, grade N42 NdFeB permanent magnet (KJ Magnetics, RA2ADIA, outer diameter=15.875 mm, inner diameter=3.175 mm, and length=15.875 mm), that rotates on a shaft (McMaster-Carr #1265K11) supported by 2 stainless steel bearings 134 (McMaster-Carr #57155K339). The rotating magnet assembly 122 may be housed within a 3D printed poly lactic acid (PLA) shell that is wrapped by 24 turns of AWG32 wire per phase.

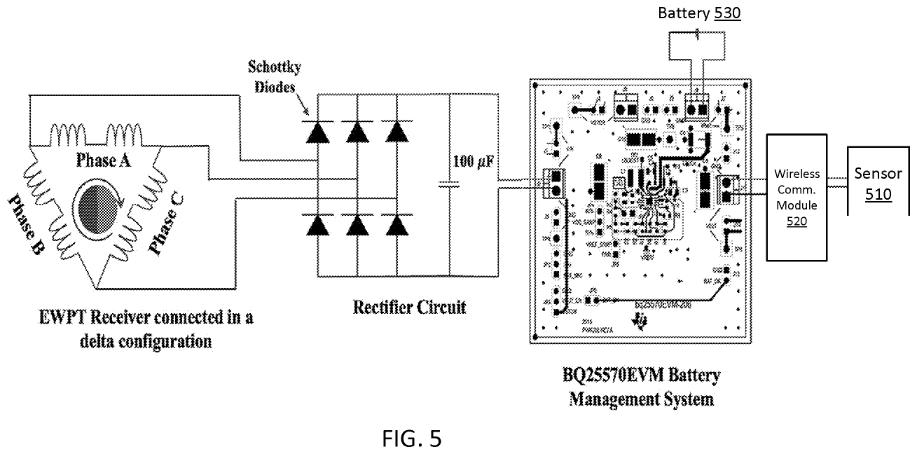

For experimental testing of this particular implementation, an arbitrary waveform generator (Agilent 33220A) 112 is utilized to produce an AC voltage, and is amplified by a Crown XLS2500 linear power amplifier 114. Current fed into the transmitter coil 116 is measured using a Tektronix TCP312A current probe connected to a Tektronix TCPA300 current probe amplifier. The voltage across the transmitter coil 116 is monitored using a Teledyne Lecroy AP031-ND differential probe. Both current and voltage probes are connected to an Agilent DSO-X-2004A 4-channel oscilloscope for capturing and analyzing the AC current and voltage waveforms, enabling the calculation of real power input to the coil 116. The magnetic field generated by the transmitter coil 116 is measured using a Lakeshore axial Gauss probe connected to a Lakeshore 475 DSP Gaussmeter. An AC-DC rectifier involving 6 Schottky diodes and a 100 μF capacitor is used to rectify and filter the three-phase AC waveform from the receiver before feeding into a battery management system evaluation board (BQ25570EVM-206 by Texas Instruments) to which a 3.7 V 120 mAh rechargeable coin cell (EEMB LIR245) is connected. The battery management system (BMS) regulates the charge current (to the battery) while also powering an ESP-8266 Wi-Fi (Node MCU) SoC that transmits data from a capacitive soil moisture sensor (V 1.2) to an Adafruit server via a travel router (TP-Link AC750) using the MQTT network protocol. FIG. 5 shows a schematic of the buried system described above. Accordingly, in various embodiments, a buried sensor along with a wireless communication module can be powered using a rechargeable battery and used as an IoT node to transmit sensor reading data via Wi-Fi to a cloud platform through a gateway system (e.g., Adafruit IO server).

The battery management system provides intelligent voltage monitoring of the battery. In the experimental setup, the coil transmitter 110 powers the “buried” EWPT receiver 120 and corresponding electronics positioned beneath a 13-cm thick layer of topsoil. To mimic an in-field system (like shown in FIGS. 2-3), the coil transmitter 110 is 17 cm above the soil corresponding to a 30 cm transmission distance between the transmitter 110 and the receiver 120. The receiver, rectifier circuit, BMS, coin rechargeable battery, and ESP-8266 are all housed in a 3D-printed PLA container for in-lab testing.

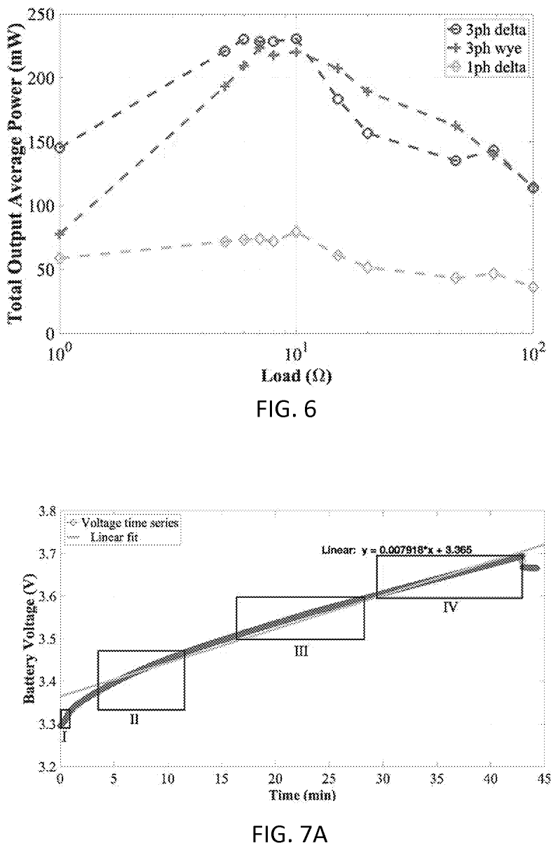

For power measurements, at 30 cm from the transmitter coil, the EWPT receiver was evaluated with resistive loads under three different loading configurations: three phase delta with a single load, and fully loaded three-phase delta and three-phase wye configurations. FIG. 6 shows the maximum power output for the fully loaded configurations were about 2.8× times higher than the single-loaded configuration, indicating higher normalized power densities for fully loaded three-phase configurations. With the goal to deliver maximum power, the fully loaded three-phase delta configuration was further explored.

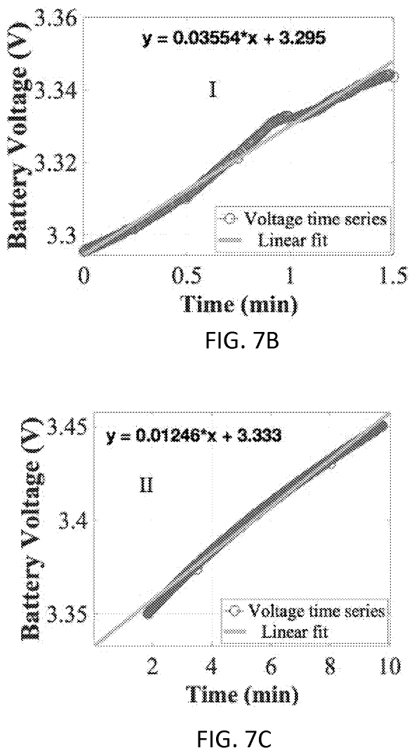

The receiver was driven by sweeping frequencies from 10 Hz to 120 Hz, providing a DC voltage of 900 mV at the input of the BMS. The current to the battery was limited to the default value of 100 mA set by the BMS. Positioned at 30 cm, at an incident field of 0.224 m TRMS (Pin=12.7 W), the receiver successfully charged the coin battery. FIG. 7A illustrates the battery voltage increasing from 3.3 V to 3.7 V over a 43 minute charging time across different sections of the charging time (sections I, II, III, and IV). FIG. 7B (Section I) illustrates an increase in voltage from 3.3 V to 3.35 V in 1.5 minutes during the ramp up stage. At 120 Hz steady state rotation, the battery recharge times are as follows: from 3.35 V to 3.45 V (Section II) in 8 minutes, 3.5 V to 3.6 V (Section III) in 13 minutes and 3.6 V to 3.7 V (Section IV) in 14 minutes. This indicates increasing recharge times as the battery approaches a fully charged state, as shown in FIGS. 7C-7E.

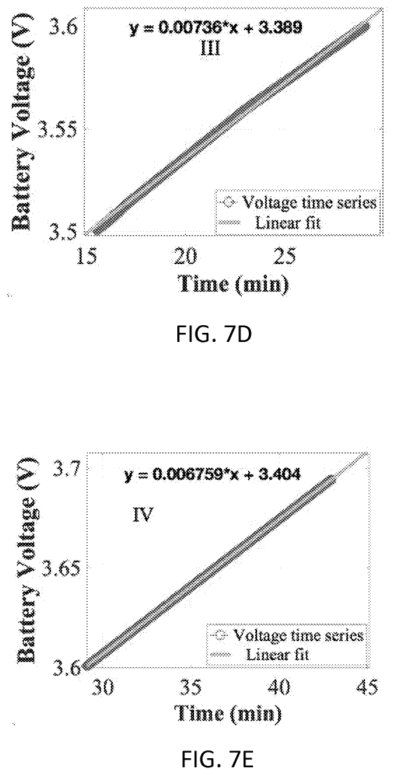

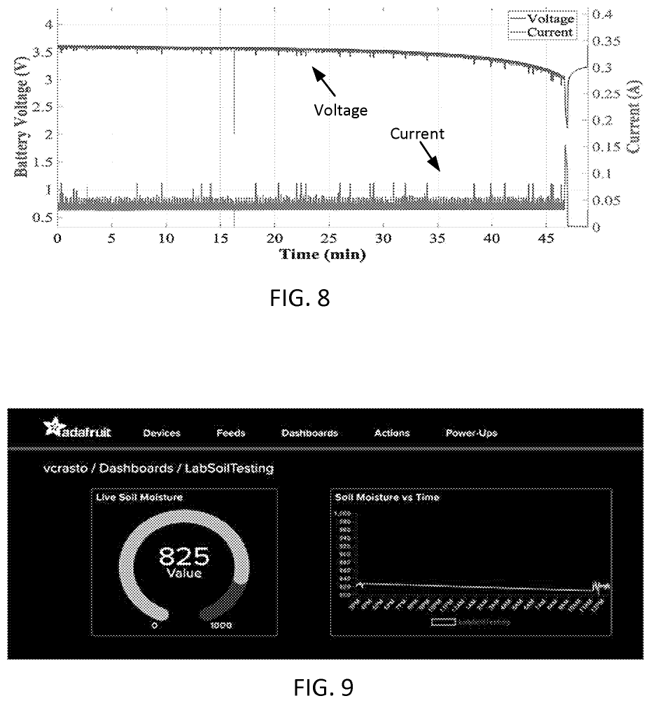

For this particular implementation, the ESP-8266 was programmed to upload soil moisture data every 20 seconds. FIG. 8 illustrates the voltage and current consumption of the ESP-8266 throughout the 47 minute discharging cycle until the battery voltage dropped to 3.2 V at which point, the battery management system cut power to the ESP-8266 to prevent excessive battery depletion and extend battery life. FIG. 9 shows a dashboard interface (e.g., Adafruit IO dashboard) of a soil moisture reading for an exemplary EWPT system in accordance with various embodiments of the present disclosure.

Alternatively, in various embodiments, the EWPT receiver 120 comprises a five-phase EWPT receiver (Receiver C). For one set of prototypes, off-the-shelf commercial coreless DC motors were modified into EWPT receivers containing a permanent magnet and 5 phase windings connected internally. Different configurations of loading the receiver were attempted and were tested at distances at 15 cm from the transmitter. With a normalized power density (NPD) of 509 mW/cm3·mT2, this is the highest reported value for an EWPT receiver. Power transfer with multiple receivers placed at 15 cm from the transmitters was demonstrated. In various embodiments, a 3D-printed custom connector was used to access all five phases of the receiver 120.

During experimental testing of Receivers A, B, and C, the three receivers illustrate identical pull-out frequencies and operating ranges, the receivers demonstrating distinct output power bands with Receiver B demonstrating 975 mW at 30 cm, and Receiver A producing 250 mW at 30 cm. Receiver C, due to a lightweight permanent magnet and frictionless bearings, makes it responsiveness at weaker fields, thus producing 2.5 mW up to 50 cm.

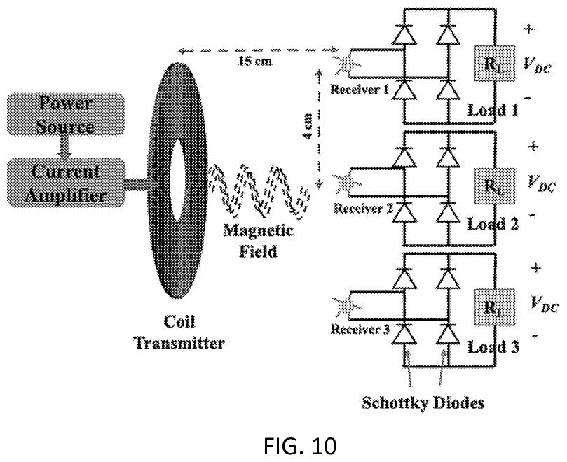

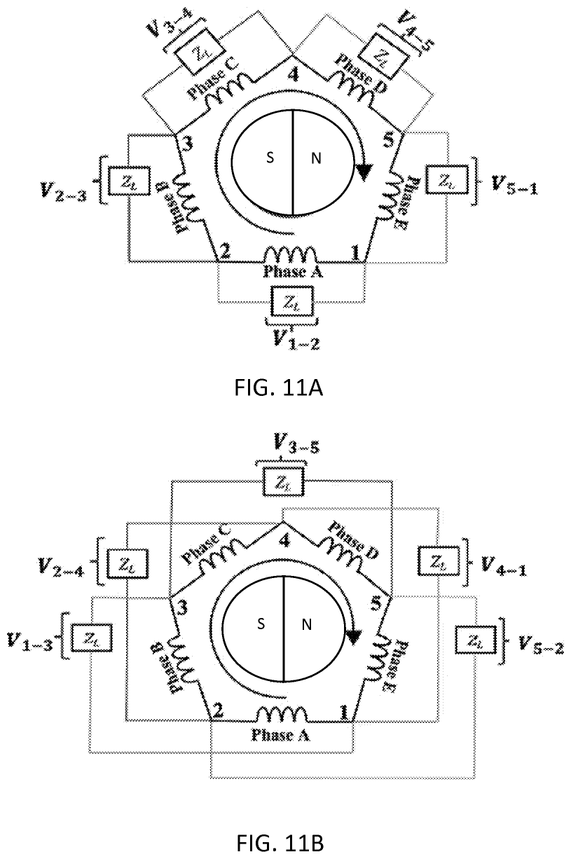

FIG. 10 shows a schematic of an exemplary EWPT system with three five-phase receivers connected to identical rectifier circuits and loads. Three different loading configurations were experimented, as illustrated in FIGS. 11A-11C. Configurations in FIGS. 11A, 11B, and 11C involve loading the receiver with five identical loads across each phase spanning 72 degrees (FIG. 11A, configuration A), loading the receiver with five identical loads across two phases spanning 144 degrees (FIG. 11B, configuration B) and loading the receiver with a single load across two phases spanning 144 degrees (FIG. 11C, configuration C) respectively. The last configuration is further explored with rectification.

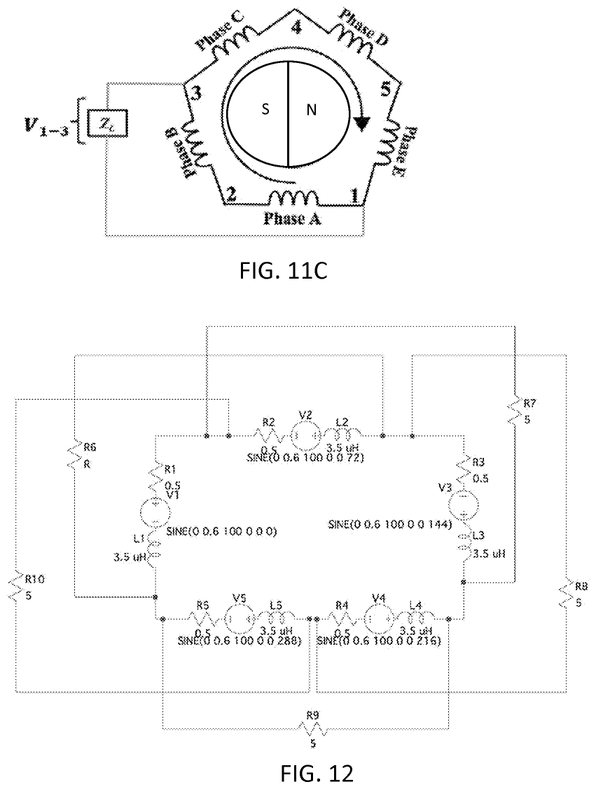

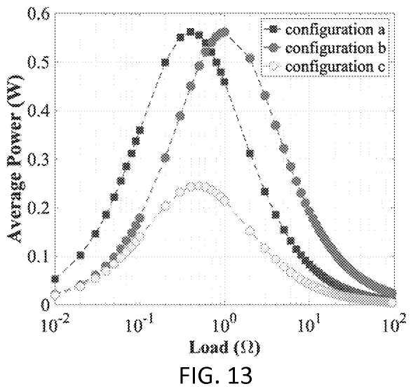

FIG. 12 shows an equivalent circuit model of the receiver, where each phase is shifted 72° relative to its neighboring phase. Phase resistance and inductance of 0.5Ω and 3.5 μH, respectively, were inferred by terminal measurements at 1 KHz using an LCR meter. In operation, the rotating magnet synchronously induces five voltage phases that increase proportional to rotation speed. Hence, for maximum power transfer, it is desirable to operate the receiver at the highest rotational speed, but the maximum achievable speed is limited by the pull-out frequency, above which the magnet loses synchronization with the excitation field and no longer spins. As shown in FIG. 13, simulations indicate that, for a given rotational speed, the receiver loaded spanning two phases (144° between nodes 1-3, 2-4, 3-5, . . . ) produced similar output power figures when compared to loads spanning one phase (72° between nodes 1-2, 2-3, 3-4, . . . ).

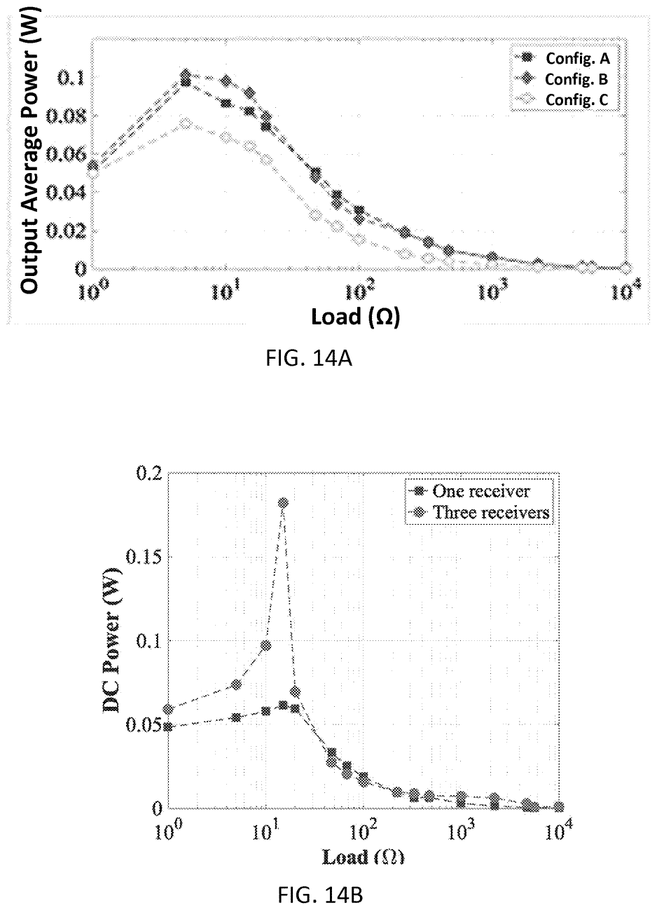

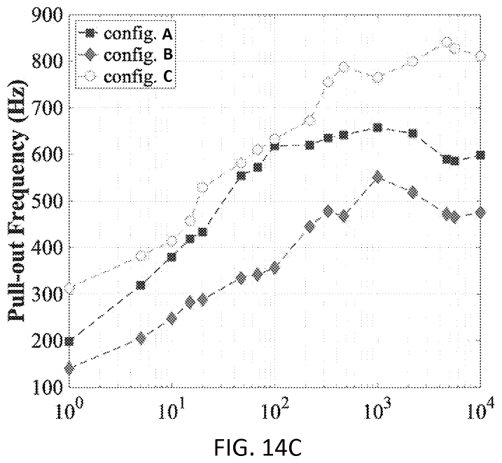

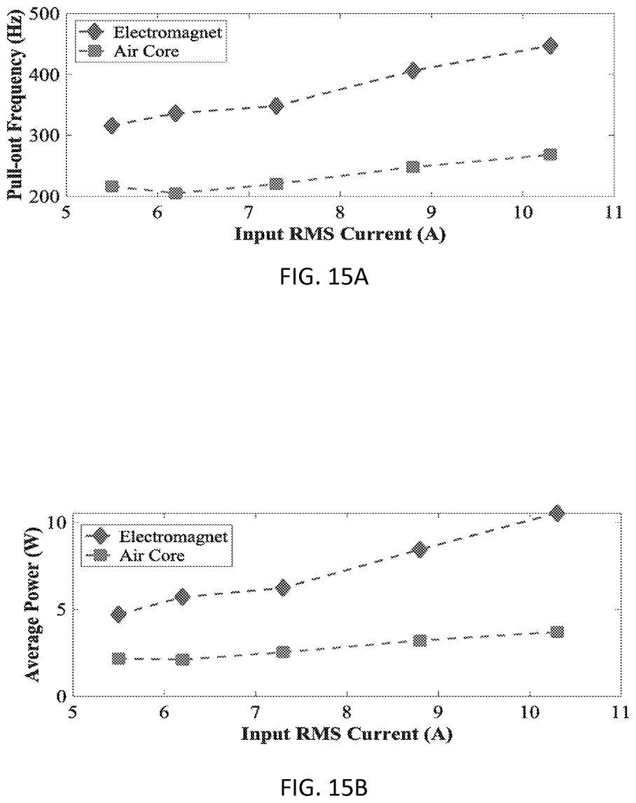

For the five-phase receiver, at a distance of 15 cm, AC power of 75 mW was delivered across a single load at 382 Hz and AC power of 101 mW was delivered across five identical loads at 210 Hz, as demonstrated in FIGS. 14A-14B. After rectification, three receivers placed 4 cm apart produced a maximum DC power of 182 mW, three times more DC power, confirming the possibility of power scaling.

In particular, during wireless power testing, a sinusoidal current (15.4 ARMS) is injected into a pancake transmitter coil (diameter=30 cm; thickness=1.7 cm; Number of turns=350) producing a time varying magnetic field that induces a rotation of the receiver magnet. As the frequency is swept from 22 Hz to the pull-out frequency at a ramp rate of 5 Hz/s, the receiver magnet begins to oscillate in a random motion and attempts to synchronize with the frequency of the incident B-field. The magnet achieves synchronization with the transmitter at frequencies between 60-70 Hz. The rotating magnet induces a sinusoidal voltage in the five coil windings (each phase shifted by) 72°. All receiver testing was performed at a distance of 15 cm from the transmitter.

AC power was evaluated for three different resistive loading configurations: configuration A consisted of five identical loads spanning one phase (72° between nodes 1-2, 2-3, 3-4, . . . ), configuration B consisting of five identical loads spanning two phases (144° between nodes 1-3, 2-4, 3-5, . . . ) and configuration C that involved one load spanning two phases (144° between nodes 1-3). The load resistances were varied from 1Ω to 10 kΩ, and the output power was found to be higher for configurations A and B compared to configuration C. At a given frequency, the power output was found to be higher for configuration B compared to configuration A.

While configuration C, connected to a single load, permitted higher speeds due to its lower electromechanical damping (75 mW at 382 Hz), configurations A and B using quintuple loads produced larger total powers of 97 mW and 101 mW at speeds 312 Hz and 210 Hz, respectively, as seen in FIGS. 14A and 14C. The power transfer efficiencies for configurations A, B and C were found to be 3.6%, 3.7% and 3.3% respectively. The power densities and normalized power densities for each configuration are given in Table 1 (below).

TABLE 1

| | Max. AC | Power | Normalized |
|---|---|---|---|
| Config. | Power | Density | Power Density |
| Type | (mW) | (mW/cm³) | (mW/cm³ · mT²) |
| A | 97 | 85.5 | 562 |
| B | 101 | 89 | 239 |
| C | 75 | 66 | 509 |

Although configurations A and B produced similar maximum power levels, configuration A generated the same power at a higher frequency. It is important to note that although simulations using LT Spice indicated that for a given speed, the power produced by configuration B was higher than configuration A, the equivalent circuit model does not simulate the pull-out frequency and hence maximum power output for the respective configurations.

Next, AC-DC rectification was explored for configuration C for simplicity. The singly loaded receiver (configuration C) was connected to a full wave rectifier constructed using four Schottky diodes. The rectified DC power to the resistive load was a maximum of 61 mW, achieved at 547 Hz, which is only 18% less than direct connection to a resistive load without the rectifier.

To investigate power transmission to multiple receivers, three receivers 120 (loaded in a 144° configuration) were placed 4 cm apart and at 15 cm from the transmitter 110. These receivers were each connected to identical full-wave rectifier circuits constructed using four Schottky diodes and further connected across three identical load resistors to measure total DC power, as shown in FIG. 10. The cluster of receivers generated a maximum combined DC power of 182 mW at 490 Hz, almost three times the power from a single receiver, as shown in FIG. 14B.

While the singly loaded receiver (configuration C) permitted higher speeds (75 mW at 382 Hz), the quintopoly loaded receiver (configuration B) produced 35% more power, 101 mW at 210 Hz. After rectification using configuration C, the DC power to the resistive load was a maximum of 61 mW, achieved at 547 Hz which is only 18% less than direct connection to a resistive load without the rectifier. With the power transfer to the multiple receivers, the cluster of receivers generated a maximum combined DC power of 182 mW at 490 Hz compared to 61 mW from a single receiver.

The transmitter plays a central role in enabling efficient and long-range electrodynamic wireless power transfer (EWPT), as it is responsible for generating the magnetic fields that interact with the receiver to induce electrical energy. Rotating magnet transmitters, in contrast to coil-based systems, offer a fundamentally different mechanism for generating time-varying magnetic fields in electrodynamic wireless power transfer (EWPT). Rather than relying on alternating currents through stationary windings, rotating magnet transmitters employ the mechanical rotation of permanent magnets to produce a rotating magnetic field in space. One of the key advantages of rotating magnets lies in their ability to generate strong magnetic fields without the significant power overhead typically associated with coil-based transmitters.

While rotating magnet transmitters have demonstrated higher efficiency and output power than conventional air-core coil transmitters, they come with their own set of trade-offs. We now know that achieving effective power transfer over longer distances requires that transmitters generate stronger magnetic fields at those distances. For coil-based transmitters, this can be achieved relatively easily by adjusting the coil geometry (e.g., increasing the number of turns, diameter, or layering) or by driving it with higher currents, both of which increase the field strength without significantly altering the physical footprint of the system. The currents injected into these coils are limited due to ohmic heating. In contrast, for rotating magnet transmitters, increasing field strength at extended distances demands the use of stronger or larger permanent magnets. Because magnetic field strength from a dipole source decays rapidly with distance (1/d³) maintaining sufficient field levels at 10-30 cm away necessitates a substantial increase in the volume or grade of the magnet. This scaling comes at a cost-larger magnets not only increase the system's weight and mechanical load, but also pose practical challenges. A significantly large permanent magnet can become dangerous, as it continuously possesses a strong magnetic field, which can unintentionally attract nearby ferromagnetic objects and pose safety risks to equipment and personnel. Furthermore, the field cannot be turned off, making the system less practical for environments that require precise field controls or safe idle states. In addition to this critical limitation, rotating magnet systems also suffer from mechanical wear, motor inefficiencies, and bearing friction, particularly when operated at high speeds needed to drive electrodynamic receivers. These considerations motive the need for alternative transmitter architectures that offer strong, controllable fields without the safety and scalability concerns associated with large rotating magnets.

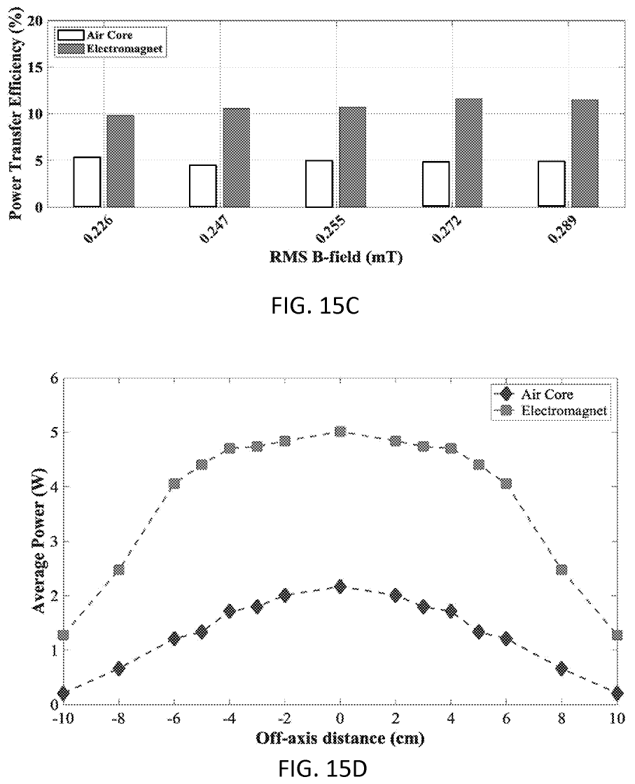

Accordingly, in various embodiments, an electromagnet transmitter may be deployed, which combines the geometric simplicity of a coil-based system with the enhanced field strength provided by the soft magnetic core, almost imitating a permanent magnet. Electromagnets offer a key advantage of field controllability. They can be energized or de-energized as needed while also improving magnetic flux concentration and power transfer performance compared to air-core coils at the cost of added complexity and material losses. For wireless power transfer characterization of an electromagnet transmitter in comparison with an air-core transmitter, a receiver was driven by both the air-core coil transmitter and the electromagnet transmitter (having a low-carbon steel based electromagnet. As shown in FIGS. 15A-15B, at identical input currents, the electromagnet transmitter with its stronger fields, drives the receiver to higher frequencies as indicated by the increased pull-out frequencies up to 447 Hz for an input current of 10.3 A_{RMS} compared to 268 Hz for the air-core coil for the same input current. This corresponds to an average power output of 10.5 W at 29% efficiency for the electromagnet transmitter compared to 3.7 W at 19% efficiency for the air-core coil, demonstrating a 2.8 times improvement over the air-core coil.

Furthermore, the power transfer efficiencies between the air-core coil and the electromagnet transmitters using the soft iron core, as seen in FIG. 15C, were compared at similar field amplitudes. At a B-field of 0.255 mT_{RMS} at which the receiver experiences pull-out, the power transfer efficiency achieved for air-core coil transmitter was 5% compared to 11% demonstrated by the electromagnet transmitter, indicating 2.2 times higher power transfer efficiencies displayed by the electromagnet transmitter, making it a better alternative to the air-core coil transmitter, in certain contexts.

The impact of transmitter types on off-axis power transfer was systematically evaluated, demonstrating the electromagnet transmitter's superior performance over the air-core coil transmitter. As illustrated in FIG. 15D, power transfer was characterized by the air-core coil transmitter delivering 210 mW at 75 Hz to a rotating magnet receiver positioned 10 cm away at-an off-axis distance of 10 cm. Under the same conditions, when excited by the electromagnet transmitter, the receiver produced a remarkable 1.3 W at 166 Hz at an off-axis distance of 10 cm, demonstrating the electromagnet's superior power transfer capability, even at off-axis distances.

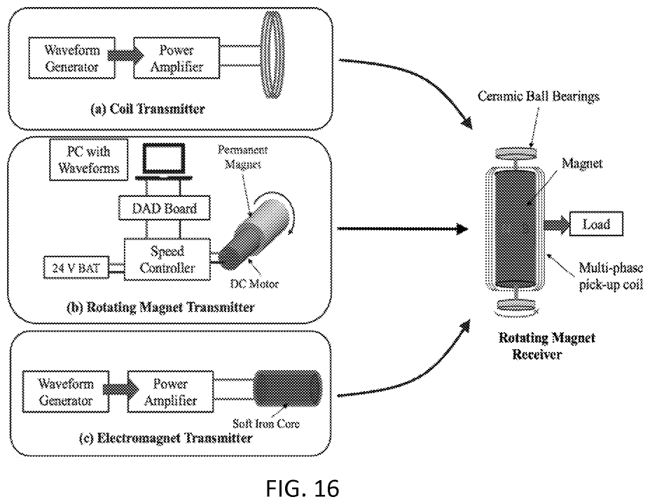

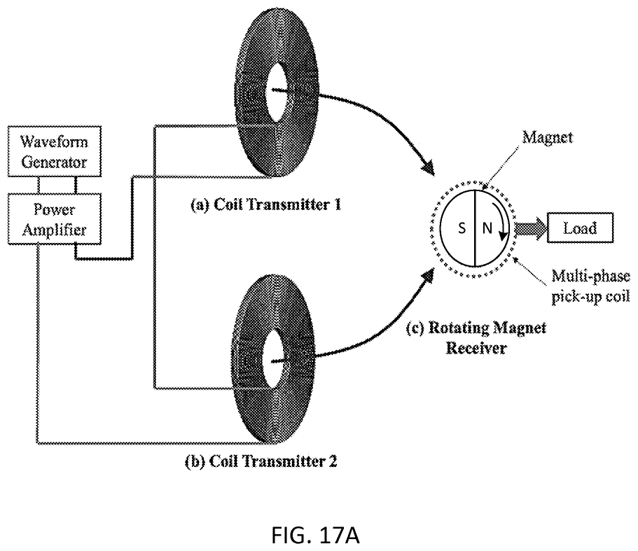

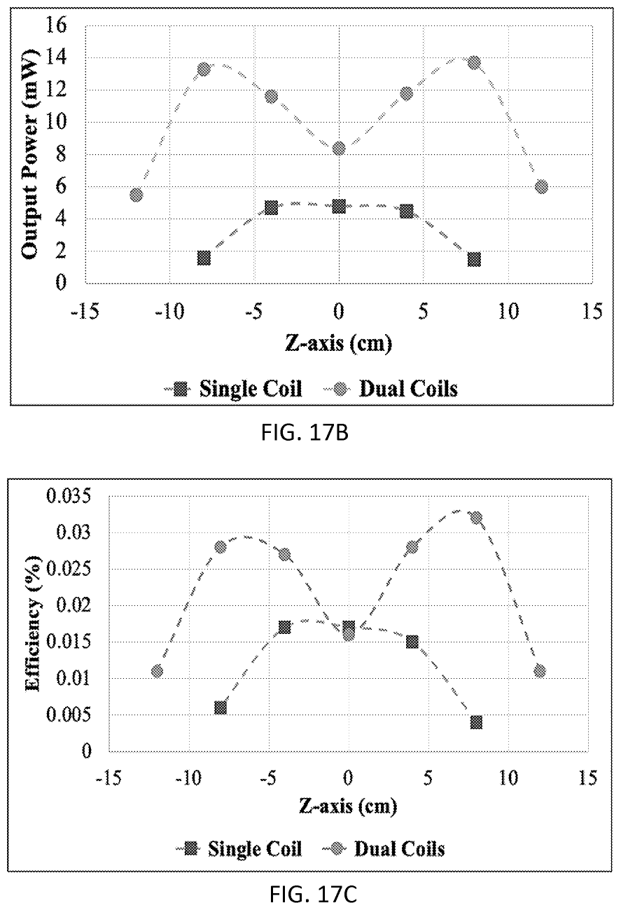

Thus, in accordance with various embodiments of the present disclosure, a rotating magnetic receiver can be driven either by (a) a coil transmitter, (b) a rotating magnet transmitter, or (c) an electromagnet transmitter, as shown in FIG. 16. Moreover, with respect to coil transmitters, single coil transmitters or multiple coil transmitters may be deployed, in various embodiments, for electrodynamic wireless power transfer. For example, a single-coil transmitter produces strong magnetic coupling along its central axis, but its performance degrades rapidly under spatial misalignment. In contrast, a dual-coil configuration driven with alternating polarity generates a U-shaped magnetic field distribution that reduces coupling at the symmetry plane while extending the effective spatial coverage. As illustrated in FIG. 17A, the dual-coil configuration contains two coils ((a) Coil Transmitter 1 and (b) Coil Transmitter 2) driven with opposite polarity, producing a U-shaped magnetic field distribution analogous to that of a U-shaped permanent magnet. In one implementation, each coil had an identical design (N=110 turns) with dimensions of 7.5 cm outer diameter, 1.5 cm inner diameter, and a length of 1.7 cm.

In an exemplary dual-coil configuration, the two coils are connected in series with alternating polarity spaced 16 cm apart. For experiments involving both transmitters, the receiver was positioned at multiple locations in the x-z plane, spanning z-axis separations from −12 cm to 12 cm and x-axis offsets from 4 cm to 12 cm to map performance across the measurement region. The receiver was terminated with a 5Ω resistive load to characterize power performance.

Output power was subsequently measured to quantify the effective energy transfer to the rotating-magnet receiver, as shown in FIG. 17B. With the single-coil transmitter, maximum power occurred along the central axis, but decreased rapidly as the receiver was displaced laterally or axially, consistent with the dipole-like field distribution of a single coil. In contrast, the dual-coil transmitter, with coils separated by 16 cm, produced a U-shaped power distribution characterized by reduced output at the symmetry plane and enhanced power near the coil faces. At a separation of x=12 cm, the dual-coil configuration delivered 13.7 mW (z=+8 cm) compared to 4.8 mW (z=0 cm) for the single coil-transmitter, corresponding to a 2.8× improvement. FIG. 17C presents a comparison of measured efficiencies for the single- and dual-coil transmitters across the x-z plane. Both configurations achieved peak efficiencies of 4.1% at x=4 cm, but performance declined rapidly at off-axis positions. At x=12 cm, the dual-coil configuration maintained higher efficiencies, reaching a peak of 0.032% at z=+8 cm, compared to 0.017% at z=0 cm for the single-coil transmitter, representing a 1.8 times improvement.

In summary, EWPT transmitters and receivers of various embodiments of the present disclosure are suited to recharge batteries and power up sub-surface sensors and communication devices. It should be emphasized that the above-described embodiments of the present disclosure are merely possible examples of implementations set forth for a clear understanding of the principles of the disclosure. Many variations and modifications may be made to the above-described embodiment(s) without departing substantially from the principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

It should be emphasized that the above-described embodiments of the present invention are merely possible examples of implementations, merely set forth for a clear understanding of the principles of the disclosure. Many variations and modifications may be made to the above-described embodiment(s) without departing substantially from the principles of the invention. All such modifications and variations are intended to be included herein within the scope of this disclosure.

We claim:

1. A wireless power transmission system comprising:
an above-ground wireless power transmitter comprising a waveform generator and a coil transmitter that is configured to generate a time-varying field; and
an underground electromechanical receiver comprising a permanent magnet that oscillates in response to the time-varying field that results in electricity being generated using electrodynamic transduction and being supplied to an underground electrical device that is coupled to the underground electromechanical receiver.

2. The system of claim 1, further comprising a plurality of underground electromechanical receivers, wherein the plurality of underground electromechanical receivers include the underground electromechanical receiver.

3. The system of claim 1, wherein the underground electrical device comprises a rechargeable battery.

4. The system of claim 1, wherein the underground electrical device comprises a soil moisture sensor.

5. The system of claim 1, wherein the underground electromechanical receiver has five-phase windings.

6. The system of claim 1, wherein the underground electromechanical receiver has three-phase windings.

7. The system of claim 1, wherein the above-ground wireless power transmitter is mounted on a vehicle that is configured to traverse a field, wherein the underground electromechanical receiver is buried in the field.

8. The system of claim 7, wherein the time-varying field is transmitted over a distance of 10 cm to 30 cm from the above-ground wireless power transmitter to the underground electromechanical receiver.

9. The system of claim 1, wherein the underground electromechanical receiver further comprises a pickup coil and a rechargeable battery, wherein oscillation of the permanent magnet induces an alternating voltage waveform in the pickup coil that is input to the rechargeable battery.

10. A wireless power transmission method comprising:
coupling a pickup coil of an electromechanical receiver to a rechargeable battery, wherein the electromechanical receiver further comprises a permanent magnet that oscillates in response to a time-varying field that results in electricity being generated using electrodynamic transduction and being supplied to the rechargeable battery via the pickup coil;
burying the electromechanical receiver and the rechargeable battery underground;
generating the time-varying field with an above-ground wireless power transmitter; and
charging the rechargeable battery that is buried underground.

11. The method of claim 10, further comprising powering an underground sensor with the rechargeable battery.

12. The method of claim 11, further comprising:
powering a wireless communication module with the rechargeable battery and
transmitting sensor readings from the underground sensor using the wireless communication module to an above ground communication gateway device.

13. The method of claim 10, wherein the electromechanical receiver has five-phase windings.

14. The method of claim 10, wherein the electromechanical receiver has three-phase windings.

15. The method of claim 10, further comprising mounting the above-ground wireless power transmitter on a vehicle that traverse a field, wherein the electromechanical receiver is buried in the field.
